SEQUENCE LISTING (16 sequences)

SEQ ID NO: 1 - protein, 638 aa, Severe Acute Respiratory Virus
GAVTRYVDNNFCGPDGYPLDCIKDFLARAGKSMCTLSEQLDYIESKRGVYCCRDHEHEIAWFTERSDKSYEHQTPFEIKSAKKFDTFKGECPKFVFPLNSKVKVIQPRVEKKKTEGFMGRIRSVYPVASPQECNNMHLSTLMKCNHCDEVSWQTCDFLKATCEHCGTENLVIEGPTTCGYLPTNAVVKMPCPACQDPEIGPEHSVADYHNHSNIETRLRKGGRTRCFGGCVFAYVGCYNKRAYWVPRASADIGSGHTGITGDNVETLNEDLLEILSRERVNINIVGDFHLNEEVAIILASFSASTSAFIDTIKSLDYKSFKTIVESCGNYKVTKGKPVKGAWNIGQQRSVLTPLCGFPSQAAGVIRSIFARTLDAANHSIPDLQRAAVTILDGISEQSLRLVDAMVYTSDLLTNSVIIMAYVTGGLVQQTSQWLSNLLGTTVEKLRPIFEWIEAKLSAGVEFLKDAWEILKFLITGVFDIVKGQIQVASDNIKDCVKCFIDVVNKALEMCIDQVTIAGAKLRSLNLGEVFIAQSKGLYRQCIRGKEQLQLLMPLKAPKEVTFLEGDSHDTVLTSEEVVLKNGELEALETPVDSFTNGAIVGTPVCVNGLMLLEIKDKEQYCALSPGLLATNNVFRLKG

SEQ ID NO: 2 - protein, 422 aa, Severe Acute Respiratory Virus
MSDNGPQSNQRSAPRITFGGPTDSTDNNQNGGRNGARPKQRRPQGLPNNTASWFTALTQHGKEELRFPRGQGVPINTNSGPDDQIGYYRRATRRVRGGDGKMKELSPRWYFYYLGTGPEASLPYGANKEGIVWVATEGALNTPKDHIGTRNPNNNAATVLQLPQGTTLPKGFYAEGSRGGSQASSRSSSRSRGNSRNSTPGSSRGNSPARMASGGGETALALLLLDRLNQLESKVSGKGQQQQGQTVTKKSAAEASKKPRQKRTATKQYNVTQAFGRRGPEQTQGNFGDQDLIRQGTDYKHWPQIAQFAPSASAFFGMSRIGMEVTPSGTWLTYHGAIKLDDKDPQFKDNVILLNKHIDAYKTFPPTEPKKDKKKKTDEAQPLPQRQKKQPTVTLLPAADMDDFSRQLQNSMSGASADSTQA

SEQ ID NO: 3 - protein, 932 aa, Severe Acute Respiratory Virus
SADASTFLNRVCGVSAARLTPCGTGTSTDVVYRAFDIYNEKVAGFAKFLKTNCCRFQEKDEEGNLLDSYFVVKRHTMSNYQHEETIYNLVKDCPAVAVHDFFKFRVDGDMVPHISRQRLTKYTMADLVYALRHFDEGNCDTLKEILVTYNCCDDDYFNKKDWYDFVENPDILRVYANLGERVRQSLLKTVQFCDAMRDAGIVGVLTLDNQDLNGNWYDFGDFVQVAPGCGVPIVDSYYSLLMPILTLTRALAAESHMDADLAKPLIKWDLLKYDFTEERLCLFDRYFKYWDQTYHPNCINCLDDRCILHCANFNVLFSTVFPPTSFGPLVRKIFVDGVPFVVSTGYHFRELGVVHNQDVNLHSSRLSFKELLVYAADPAMHAASGNLLLDKRTTCFSVAALTNNVAFQTVKPGNFNKDFYDFAVSKGFFKEGSSVELKHFFFAQDGNAAISDYDYYRYNLPTMCDIRQLLFVVEVVDKYFDCYDGGCINANQVIVNNLDKSAGFPFNKWGKARLYYDSMSYEDQDALFAYTKRNVIPTITQMNLKYAISAKNRARTVAGVSICSTMTNRQFHQKLLKSIAATRGATVVIGTSKFYGGWHNMLKTVYSDVETPHLMGWDYPKCDRAMPNMLRIMASLVLARKHNTCCNLSHRFYRLANECAQVLSEMVMCGGSLYVKPGGTSSGDATTAYANSVFNICQAVTANVNALLSTDGNKIADKYVRNLQHRLYECLYRNRDVDHEFVDEFYAYLRKHFSMMILSDDAVVCYNSNYAAQGLVASIKNFKAVLYYQNNVFMSEAKCWTETDLTKGPHEFCSQHTMLVKQGDDYVYLPYPDPSRILGAGCFVDDIVKTDGTLMIERFVSLAIDAYPLTKHPNQEYADVFHLYLQYIRKLHDELTGHMLDMYSVMLTNDNTSRYWEPEFYEAMYTPHTVLQ

SEQ ID NO: 4 - DNA, 23 bp, Artificial Sequence
  Description of Artificial Sequence Synthetic Primer
  modified_base (1): deoxy 5-methyl isocytidine
natcacccgcgaagaagctattc

SEQ ID NO: 5 - DNA, 22 bp, Artificial Sequence
  Description of Artificial Sequence Synthetic Primer
agccctctacatcaaagccaat

SEQ ID NO: 6 - DNA, 22 bp, Artificial Sequence
  Description of Artificial Sequence Synthetic Primer
  modified_base (1): deoxy 5-methyl isocytidine
ncaaagacaacgtcatactgct

SEQ ID NO: 7 - DNA, 23 bp, Artificial Sequence
  Description of Artificial Sequence Synthetic Primer
ttttgtcctttttaggctctgtt

SEQ ID NO: 8 - DNA, 21 bp, Artificial Sequence
  Description of Artificial Sequence Synthetic Primer
  modified_base (1): deoxy 5-methyl isocytidine
ncaagggaaagcccgtaaaag

SEQ ID NO: 9 - DNA, 19 bp, Artificial Sequence
  Description of Artificial Sequence Synthetic Primer
agcctgtgagggaaaacca

SEQ ID NO: 10 - DNA, 18 bp, Unknown Organism
  Description of Unknown Organism Exemplary Control Sequence
  modified_base (2): deoxy 5-methyl isocytidine
tngcctgctgtgctgtgt

SEQ ID NO: 11 - DNA, 14 bp, Unknown Organism
  Description of Unknown Organism Exemplary Control Sequence
tcgtgcggtgcgtc

SEQ ID NO: 12 - DNA, 59 bp, Unknown Organism
  Description of Unknown Organism Exemplary Control Sequence
atcacccgcgagagctattcgtcacgttcgtgcttggattggctttgatgtagagggct

SEQ ID NO: 13 - DNA, 80 bp, Unknown Organism
  Description of Unknown Organism Exemplary Control Sequence
caaagacaacgtcatactgctgaacaagcacattgacgcatacaaaacattcccaccaacagagcctaaaaaggacaaaa

SEQ ID NO: 14 - RNA, 88 nt, Unknown Organism
  Description of Unknown Organism Exemplary Control Sequence
gggcaagggaaagcccguaaaaggugcuuggaacauuggacaacagagaucaguuuuaccaccacugugugguuuucccucacaggcu

SEQ ID NO: 15 - RNA, 30 nt, Unknown Organism
  Description of Unknown Organism Exemplary Control Sequence
ucgugcggugcgucacacagcacagcaggc

SEQ ID NO: 16 - RNA, 143 nt, Unknown Organism
  Description of Unknown Organism Exemplary Control Sequence
ugcauccaacgcguugggagcucucccauauggucgaccugcaggcggcgcacuagugauacgcugcugugcugugugacgcaccgcacgaaaucccgcggccauggcggccgggagcaugcgacgucgggcccaauucgccc

(19) United States
(12) Patent Application Publication
(10) Pub. No.: US 2007/0059686 A1
(43) Pub. Date: Mar. 15, 2007

(54) Materials and methods for the detection of severe acute respiratory syndrome virus (SARS)

(75) Inventors: Michael James Moser, Madison, WI (US); James R. Prudent, Madison, WI (US)

Correspondence Address: ERAGEN BIOSCIENCES, INC. C/O FOLEY & LARDNER LLP, P.O. BOX 1497, 150 E. GILMAN STREET, MADISON, WI, 53701, US

(73) Assignee: EraGen Biosciences, Inc.

(21) Appl. No.: 11/494,820

(22) Filed: Jul. 28, 2006

Related U.S. Application Data
(63) Continuation-in-part of application No. PCT/US05/02950, filed on Jan. 31, 2005.
(60) Provisional application No. 60/540,774, filed on Jan. 31, 2004.

(51) Int. Cl. C12Q 1/70; C12Q 1/68

(52) U.S. Cl. 435/5; 435/6

(57) ABSTRACT

Disclosed are methods and kits for identifying a virus in a sample, which may include a coronavirus capable of causing Severe Acute Respiratory Syndrome (“SARS”) or SARS-like symptoms. The virus may be a SARS-Associated Coronavirus (“SARS-CoV”). Typically, the methods may include reacting a mixture that includes, in addition to nucleic acid isolated from the sample, at least one oligonucleotide capable of specifically hybridizing to SARS-CoV nucleic acid where the oligonucleotide includes at least one non-natural base. In addition, the mixture may include control nucleic acid.

### CROSS-REFERENCE TO RELATED APPLICATIONS

This application is a continuation-in-part, and claims priority under 35 U.S.C. § 365(c) to international application no. PCT/US2005/002950 filed on Jan. 31, 2005, and published as WO 2005/081776 on Sep. 9, 2005; which claims priority to U.S. provisional application No. 60/540,770, filed Jan. 30, 2004, now abandoned. This application includes subject matter related to subject matter disclosed in U.S. provisional application No. 60/472,928, filed on May 23, 2003, now abandoned.

### FIELD OF THE INVENTION

The invention relates generally to the field of identifying nucleic acids. More specifically the invention generally relates to diagnostic methods that may be useful for diagnosing patients infected with an agent capable of cause Severe Acute Respiratory Syndrome (“SARS”) or SAR-like symptoms.

### BACKGROUND

As described by the Center for Disease Control (“CDC”) and the World Health Organization, (“WHO”), Severe Acute Respiratory Syndrome (“SARS”) is caused by a novel coronavirus named SARS-associated coronavirus (SARS-CoV).

The coronaviruses are enveloped, positive-strand RNA viruses with a genome size of approximately 30 kb, the largest RNA genome reported to date. Coronaviruses derived their name from the spike protein present on the outer viral membrane. These spikes give the virus a “crown-like” appearance when viewed under the microscope.

The disease SARS was first recognized as a potential global health issue in 2003, a year in which over 8000 people world-wide became ill, and over 750 died as a result of infection with SARS-CoV. The disease was quickly contained and since July 2004, there have been only four incidences of SARS infection, all of which were laboratory related exposures. SARS is currently in an “interepidemic period” (i.e., a period characterized by an absence of human chains of SARS-CoV transmission worldwide), and predictions of when, where or if the virus will re-emerge as an epidemic are difficult to make.

Researchers world-wide have worked toward developing fast and accurate laboratory diagnostic tests for SARS-CoV. Formats for these diagnostic tests include enzyme-linked immunosorbent assays (“ELISA”) as well as nucleic acid-based assays that generally include the polymerase chain reaction (“PCR”). Assays utilizing PCR, for example quantitative reverse transcription-PCR (qRT-PCR), are generally more sensitive than ELISA based assays, but are also more expensive and more difficult to conduct. See, e.g., Lau, et al., Emerging Infectious Diseases 2005, 11:7 1108-1111.

Nucleic acid based assays that include PCR have been shown to be capable of detecting SARS nucleic acid from samples such as stool, urine, blood, respiratory secretions or other body tissue. Primers, which are the key components of a PCR test, have been made publicly available by WHO network laboratories on the WHO web site. Further, ready-to-use PCR test kits containing primers and positive and negative controls have been developed. Principally, the existing PCR tests are very specific but lack sensitivity. This means that a negative test result cannot rule out the presence of SARS virus in patients. Further, contamination of samples in laboratories in the absence of laboratory quality control can lead to false positive results.

Sensitivity is critical to early SARS diagnosis. During the initial phase of SARS, viral loads are relatively low while peak viral loads are reached at 12-24 days of illness. Cheng, et al., Lancet 2004, 363:9422, 1699-1700. Thus, SARS patients in the early stages of illness may be misdiagnosed as SARS-negative, and sick individuals may inadvertently infect others before a proper diagnosis is made.

Accordingly, there is a need in the art for an efficient, sensitive and reliable assay to detect agents that may cause SARS or SARS-like symptoms.

### SUMMARY

The methods and kits described herein relate to detecting viral infections in mammals. In some aspects, the methods and kits relate to detecting coronaviruses that can cause Severe Acute Respiratory Syndrome (“SARS”) or SARS-like symptoms in mammals using nucleotides or oligonucleotides that include at least one non-natural nucleobase.

One method for detecting SARS virus in a mammal may include reacting a mixture which includes SARS virus nucleic acid isolated from a sample and at least one oligonucleotide that is capable of specifically hybridizing to the viral nucleic acid, where the oligonucleotide comprises at least one non-natural nucleotide. Other methods for detecting SARS virus may include reacting a mixture including SARS virus nucleic acid isolated from a sample, a control nucleic acid, and two pairs of oligonucleotides. In some methods, the first pair of oligonucleotides may be capable of hybridizing to the viral nucleic acid, and the second pair of oligonucleotides may be capable of hybridizing to the control nucleic acid, and at least one oligonucleotide of each pair of oligonucleotides may include a label that is different from the label of the other oligonucleotide pair. In another method, the viral nucleic acid and the control nucleic acid may be amplified and detected. In still another method, kits may be provided for the detection of SARS virus infection in a mammal.

The methods may be used to detect a viral agents such as SARS virus or a virus that is capable of causing SARS or SARS-like symptoms. For example, the SARS virus may include a virus having the genomic sequence provided as GenBank Accession No. NC_{—}004718, or a natural or artificial variants thereof. For example, a natural or artificial variant may include a virus having at least about 95% genomic sequence identity to the genomic sequence deposited as GenBank Accession No. NC_{—}004718. A natural or artificial variant may include a virus whose genome hybridizes to the genomic sequence deposited as GenBank Accession No. NC_{—}004718 under stringent conditions.

Some embodiments of the methods utilize at least one oligonucleotide that is capable of hybridizing to the genomic sequence of a SARS virus (e.g., under stringent conditions). The oligonucleotide may be capable of specifically hybridizing to a SARS virus nucleoprotein (“NP”) sequence, a SARS virus polymerase (“POL”) sequence or a SARS virus P65 (“P65”) sequence. For example, the oligonucleotides of the present methods may be capable of hybridizing to a SARS virus nucleotide sequences that encodes one or more of the following sequences represented by SEQ ID NOS. 1-3.

In some methods, the reaction mixture may include at least two oligonucleotides. For example, the reaction mixture may include at least two oligonucleotides that are capable of hybridizing to the SARS virus nucleic acid and that may function as primers for the amplification of SARS virus nucleic acid. In some methods, at least one of the two oligonucleotides is used as a primer and includes at least one base or nucleotide other than A, C, G, T and U; the base or nucleotide may include iC or iG (e.g., diCTP or diGTP). In some methods, at least one of the oligonucleotides used as a primer may include a first label. Suitable labels may include fluorophores and quenchers. In some methods, the reaction mixture may include a nucleotide (e.g. a non-natural nucleotide) covalently linked to a first quencher. The non-natural nucleotide may include a non-natural base such as iC or iG.

Some methods may include an oligonucleotide that functions as an internal control nucleic acid. In some embodiments, the internal control nucleic acid may be selected from sequences represented by SEQ ID NO: 12-16. In other methods, the reaction mixture may include at least two oligonucleotides capable of hybridizing to an internal control nucleic acid and that may function as primers to amplify the internal control nucleic acid. In some methods, at least one of the two oligonucleotides used as a primer for the internal control may include at least one base or nucleotide other than A, C, G, T and U. For example, the nucleotide may include iso-cytosine and/or iso-guanine (“iC” and/or “iG,” respectively). In some methods, at least one of the oligonucleotides used as a primer for the internal control may includes a second label. Suitable labels may include fluorophores and quenchers. In other methods, the reaction mixture may include a nucleotide covalently linked to a second quencher, which may the same or different as the first quencher. For example, the reaction mixture may include a non-natural nucleotide (e.g., a nucleotide having iC or iG as a base) covalently linked to a quencher.

In some methods, the first and second labels may be different. For example, in some methods the first and second quencher may be different and may be capable of quenching two different fluorophores. In other methods, the first and second quenchers may be the same and may be capable of quenching two different fluorophores.

In some embodiments, the methods may include performing reverse transcription of RNA in a sample. The methods may include performing amplification (e.g., PCR which may include RT-PCR). The methods may include hybridizing a probe to an amplified nucleic acid to detect an amplified target. For example, the methods may include performing RT-PCR followed by performing probe hybridization.

The methods described herein may include determining a melting temperature for an amplicon (e.g., amplified nucleic acid of at least one of amplified nucleic acid of SARS virus and amplified control nucleic acid). The methods may include determining a melting temperature for a nucleic acid complex that includes a labeled probe hybridized to a target nucleic acid (which may include amplified target nucleic acid). The melting temperature may be determining by exposing the amplicon or nucleic acid complex to a gradient of temperatures and observing a signal from a label. Optionally, the melting temperature may be determined by (a) reacting an amplicon with an intercalating agent at a gradient of temperatures and (b) observing a detectable signal from the intercalating agent. The melting temperature of a nucleic acid complex may be determined by (1) hybridizing a probe to a target nucleic acid to form a nucleic acid complex, where at least one of the probe and the target nucleic acid includes a label; (2) exposing the nucleic acid complex to a gradient of temperatures; and (3) observing a signal from the label.

The methods may be performed in any suitable reaction chamber under any suitable conditions. For example, the methods may be performed in a reaction chamber without opening the reaction chamber. The reaction chamber may be part of an array of reaction chambers. In some embodiments, the steps of the methods may be performed separately in different reaction chambers.

The methods may also include kits for the detection of SARS virus. In some embodiments, the kit may include a first pair of oligonucleotides capable of hybridizing to a SARS virus nucleic acid. In some embodiments, at least one oligonucleotide of the first pair may include at least one non-natural nucleotide and at least one label. In further embodiments, a kit may also include control nucleic acid and a second pair of oligonucleotides capable of hybridizing to the control nucleic acid. In some embodiments, at least one oligonucleotide of the second pair may include at least one non-natural nucleotide and a second label. In some embodiments, the first and second label may be different.

In some embodiments, the methods may be capable of detecting at least about 5 copies of SARS nucleic acid in a sample (e.g., in a sample having a volume of about 10 microliters). In other embodiments, the methods may be capable of detecting at least about 10 copies, 25 copies, 50 copies, or 100 copies of SARS nucleic acid in a sample (e.g., in a sample having a volume of about 10 microliters). In some embodiments, the methods may be capable of detecting SARS virus nucleic acid in a sample having a SARS virus concentration of at least about 10⁻⁸ PFU/ml (or at least about 10⁻⁷ PFU/ml, 10⁻⁵ PFU/ml, 10⁻³ PFU/ml, 10⁻¹ PFU/ml, or 10 PFU/ml).

### BRIEF DESCRIPTION OF THE DRAWINGS

FIG. 1 shows a MultiCode RTx system schematic. Targets may be amplified with a standard reverse primer and a forward primer that contains a single iC nucleotide and a fluorescent reporter. Amplification is performed in the presence of dabcyl-diGTP. Site-specific incorporation places the quencher in close proximity to the reporter that leads to a decrease in fluorescence.

FIG. 2a shows the standard curve for a real-time, quantitative RT-PCR assay with a SARS RNA dilution series ranging from 10⁻¹ to 10⁻⁷ copies.

FIG. 2b shows the results of a melting curve analysis of the SARS RNA dilution series.

FIG. 3a shows real-time cycle threshold data of urine samples spiked with different concentrations of SARS virus (grey lines) and RNA extracted from ten potential SARS patient urine specimens (black lines).

FIG. 3b shows post-PCR melt analysis data of urine samples spiked with different concentrations of SARS virus (grey lines) and RNA extracted from ten potential SARS patient urine specimens (black lines).

FIG. 3c shows a linear least squares fit. The black squares indicate patient RNA samples; the grey circles represent spiked urine samples.

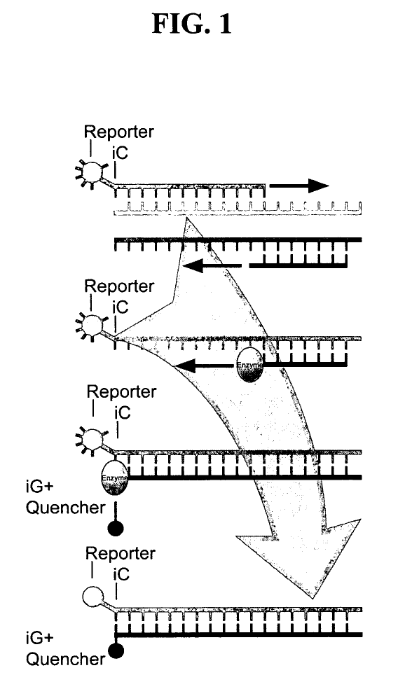

### DETAILED DESCRIPTION

Disclosed are methods and kits for detecting nucleic acids in a sample. Typically, the methods include detecting signals such as a signal emitted from a fluorophore. Also disclosed are oligonucleotides, especially primers and probes, which may be used for the detection of coronaviruses capable of causing SARS or SARS-like symptoms. The methods, kits, and oligonucleotides disclosed herein may be used to detect SARS-CoV, which has been shown to be the causative agent of SARS in humans. Some methods may be based on assay methods described in published international application WO 01/90417 and U.S. published application no. 2002/0150900, herein incorporated by reference in their entireties.

As used herein, unless otherwise stated, the singular forms “a,” “an,” and “the” includes plural reference. Thus, for example, a reference to “an oligonucleotide” includes a plurality of oligonucleotide molecules, and a reference to “a nucleic acid” is a reference to one or more nucleic acids.

As used herein, the term “sample” is used in its broadest sense. A sample may include a bodily tissue or a bodily fluid including but not limited to blood (or a fraction of blood such as plasma or serum), lymph, mucus, tears, urine, and saliva. A sample may include an extract from a cell, a chromosome, organelle, or a virus. A sample may comprise DNA (e.g., genomic DNA), RNA (e.g., mRNA), and cDNA, any of which may be amplified to provide amplified nucleic acid. A sample may include nucleic acid in solution or bound to a substrate (e.g., as part of a microarray). A sample may comprise material obtained from an environmental locus (e.g., a body of water, soil, and the like) or material obtained from a fomite (i.e., an inanimate object that serves to transfer pathogens from one host to another).

The term “source of nucleic acid” refers to any sample which contains nucleic acids (RNA or DNA). Particularly preferred sources of target nucleic acids are biological samples including, but not limited to blood, plasma, serum, saliva, cerebral spinal fluid, pleural fluid, milk, lymph, sputum and semen.

As used herein, the term “limit of detection” refers to the lowest level or amount of an analyte, such as a nucleic acid, that can be detected and quantified. Limits of detection can be represented as molar values (e.g., 2.0 nM limit of detection), as gram measured values (e.g., 2.0 microgram limit of detection under, for example, specified reaction conditions), copy number (e.g., 1×10⁵ copy number limit of detection), or other representations known in the art.

As used herein, “coronavirus” is meant to include any enveloped positive-strand RNA virus that belongs to the coronavirus family and is capable of infecting mammals or birds and causing a respiratory disease or illness.

As used herein, the terms “SARS-associated coronavirus,” “SARS-CoV,” “SARS coronavirus” and “SARS virus” are meant to include a coronavirus capable of causing SARS or SARS-like symptoms. There are many known strains of SARS coronavirus. One example of SARS-CoV is the virus isolate encoded by Genbank Accession No: NC_{—}004718. “SARS-associated coronavirus,” “SARS coronavirus” “SARS-CoV” or “SARS virus” as used herein are also meant to include any natural or artificial variants, analogs or derivatives of a SARS virus. As used herein, “variant” refers to either naturally occurring genetic mutations of the SARS virus or a recombinantly prepared variation of the SARS virus, any of which may contain one or more changes in nucleic acid sequence as compared to the SARS virus encoded by, for example, the sequence deposited as GenBank Accession No NC_{—}004718.

The SARS virus of the present methods may include nucleic acid which codes for a polymerase, a nucleocapsid protein, and a P65 protein. By way of example but not by way of limitation, an exemplary P65 protein sequence may include SEQ ID NO. 1; an exemplary nucleocapsid protein sequence may include SEQ ID NO. 2; and an exemplary polymerase sequence may include SEQ ID NO. 3. In some embodiments, the sequence of the detected SARS-CoV nucleic acid (e.g., nucleic acid of polymerase, nucleocapsid protein and/or P65 protein) may have about 95% sequence identity to any of SEQ ID NOs. 1-3. In other embodiments, the sequence of the detected SARS-CoV nucleic acid may have about 90% sequence identity to any of SEQ ID NOs. 1-3. In other embodiments, the sequence of the detected SARS-CoV nucleic acid may have about 80% sequence identity to SEQ ID NOs. 1-3.

As used herein, the term “Severe Acute Respiratory Syndrome” (“SARS”) means a viral respiratory illness caused by a coronavirus. SARS symptoms commonly include a high fever, headaches, body aches and cough. Some patients may develop diarrhea or pneumonia.

As used herein the term “isolated” in reference to a nucleic acid molecule refers to a nucleic acid molecule which is separated from the organisms and biological materials (e.g., blood, cells, serum, plasma, saliva, urine, stool, sputum, nasopharyngeal aspirates and so forth), which are present in the natural source of the nucleic acid molecule. An isolated nucleic acid molecule, such as a cDNA molecule, can be substantially free of other cellular material, or culture medium when produced by recombinant techniques, or substantially free of chemical precursors or other chemicals when chemically synthesized. In a preferred embodiment of the invention, nucleic acid molecules encoding polypeptides/proteins of the invention may also be isolated or purified. The term “isolated nucleic acid” does not include a nucleic acid that is a member of a library that has not been purified away from other library clones containing other nucleic acid molecules. Methods of nucleic acid isolation are well known in the art and may include total nucleic acid isolation/purification methods, RNA-specific isolation/purification methods or DNA-specific isolation/purification methods.

As used herein, the term “isolated virus” refers to a virus which is separated from other organisms and biological materials which are present in the natural source of the virus, e.g., biological material such as cells, blood, serum, plasma, saliva, urine, stool, sputum, nasopharyngeal aspirates, and so forth. The isolated virus can be used to infect a subject.

As used herein, the term “subject” is refers to an animal, preferably a mammal, more preferably a human. The term “subject” and “patient” may be used interchangeably.

A “mutation” is meant to encompass at least a single nucleotide variation in a nucleic acid sequence relative to the normal sequence or wild-type sequence. A mutation may include a substitution, a deletion, an inversion or an insertion. With respect to an encoded polypeptide, a mutation may be “silent” and result in no change in the encoded polypeptide sequence or a mutation may result in a change in the encoded polypeptide sequence. For example, a mutation may result in a substitution in the encoded polypeptide sequence. A mutation may result in a frameshift with respect to the encoded polypeptide sequence. For example, the SARS viruses of the present methods may be mutants as compared to the SARS virus nucleic acid of Genbank Accession No. NC_{—}004718 or other known SARS virus strains.

As used herein, the term “microarray” refers to an arrangement of a plurality of polynucleotides, polypeptides, or other chemical compounds on a substrate. The terms “element” and “array element” refer to a polynucleotide, polypeptide, or other chemical compound having a unique and defined position on a microarray.

As used herein, an oligonucleotide is understood to be a molecule that has a sequence of bases on a backbone comprised mainly of identical monomer units at defined intervals. The bases are arranged on the backbone in such a way that they can enter into a bond with a nucleic acid having a sequence of bases that are complementary to the bases of the oligonucleotide. The most common oligonucleotides have a backbone of sugar phosphate units. A distinction may be made between deoxyribonucleotides (“dNTP's”), which do not have a hydroxyl group at the 2′ position, and ribonucleotides (“NTP's”), which have a hydroxyl group in this position. Oligonucleotides also may include derivatives, in which the hydrogen of the hydroxyl group is replaced with organic groups, e.g., an allyl group.

In some embodiments, oligonucleotides as described herein may include a peptide backbone. For example, the oligonucleotides may include peptide nucleic acids or “PNA.” Peptide nucleic acids are described in WO 92/20702, which is incorporated herein by reference.

An oligonucleotide is a nucleic acid that includes at least two nucleotides. Oligonucleotides used in the methods disclosed herein typically include at least about ten (10) nucleotides and more typically at least about fifteen (15) nucleotides. Preferred oligonucleotides for the methods disclosed herein include between about 10-25 nucleotides. An oligonucleotide may be designed to function as a “primer.” A “primer” is a short nucleic acid, usually a ssDNA oligonucleotide, which may be annealed to a target polynucleotide by complementary base-pairing. The primer may then be extended along the target DNA strand by a DNA polymerase enzyme. Primer pairs can be used for amplification (and identification) of a nucleic acid sequence (e.g., by the polymerase chain reaction (PCR)). An oligonucleotide may be designed to function as a “probe.” A “probe” refers to an oligonucleotide, its complements, or fragments thereof, which is used to detect identical, allelic or related nucleic acid sequences. Probes may include oligonucleotides which have been attached to a detectable label or reporter molecule. Typical labels include fluorescent dyes, quenchers, radioactive isotopes, scintillation agents, ligands, chemiluminescent agents, and enzymes.

An oligonucleotide may be designed to be specific for a target nucleic acid sequence in a sample. For example, an oligonucleotide may be designed to include “antisense” nucleic acid sequence of the target nucleic acid. As used herein, the term “antisense” refers to any composition capable of base-pairing with the “sense” (coding) strand of a specific target nucleic acid sequence. An antisense nucleic acid sequence may be “complementary” to a target nucleic acid sequence. As used herein, “complementarity” describes the relationship between two single-stranded nucleic acid sequences that anneal by base-pairing. For example, 5′-AGT-3′ pairs with its complement, 3′-TCA-5′.

Oligonucleotides as described herein typically are capable of forming hydrogen bonds with oligonucleotides having a complementary base sequence. These bases may include the natural bases such as A, G, C, T and U, as well as artificial, non-standard, or non-natural bases such as iso-cytosine, and iso-guanine. As described herein, a first sequence of an oligonucleotide is described as being 100% complementary with a second sequence of an oligonucleotide when the consecutive bases of the first sequence (read 5′->3′) follow the Watson-Crick rule of base pairing as compared to the consecutive bases of the second sequence (read 3′->5′). An oligonucleotide may include nucleotide substitutions. For example, an artificial base may be used in place of a natural base such that the artificial base exhibits a specific interaction that is similar to the natural base.

An oligonucleotide that is specific for a target nucleic acid also may be specific for a nucleic acid sequence that has “homology” to the target nucleic acid sequence. As used herein, “homology” refers to sequence similarity or, interchangeably, sequence identity, between two or more polynucleotide sequences or two or more polypeptide sequences. The terms “percent identity” and “% identity” as applied to polynucleotide sequences, refer to the percentage of residue matches between at least two polynucleotide sequences aligned using a standardized algorithm (e.g., BLAST).

An oligonucleotide that is specific for a target nucleic acid will “hybridize” to the target nucleic acid under suitable conditions. As used herein, “hybridization” or “hybridizing” refers to the process by which an oligonucleotide single strand anneals with a complementary strand through base pairing under defined hybridization conditions. “Specific hybridization” is an indication that two nucleic acid sequences share a high degree of complementarity. Specific hybridization complexes form under permissive annealing conditions and remain hybridized after any subsequent washing steps. Permissive conditions for annealing of nucleic acid sequences are routinely determinable by one of ordinary skill in the art and may occur, for example, at 65° C. in the presence of about 6×SSC. Stringency of hybridization may be expressed, in part, with reference to the temperature under which the wash steps are carried out. Such temperatures are typically selected to be about 5° C. to 20° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Equations for calculating Tₘ and conditions for nucleic acid hybridization are known in the art.

As used herein “target” or “target nucleic acid” refers to a nucleic acid molecule containing a sequence that has at least partial complementarity with an oligonucleotide, for example a probe or a primer. A “target” sequence may include a part of a gene or genome. In some embodiments, targets are single copy sequences (e.g., there are no nucleotide sequences identical to the target sequence in the gene or genome of interest). In some embodiments, the target sequence is conserved. For example, the nucleotide sequence of the polymerase gene, the nucleocapsid gene, and the P65 gene all appear to be conserved (e.g., few nucleotide differences) among different strains of SARS-CoV. All three gene sequences may be targets for detection of SARS virus via, for example, reverse transcription, amplification, probe hybridization and other methods known in the art.

As used herein, “nucleic acid,” “nucleotide sequence,” or “nucleic acid sequence” refer to a nucleotide, oligonucleotide, polynucleotide, or any fragment thereof and to naturally occurring or synthetic molecules. These terms also refer to DNA or RNA of genomic or synthetic origin which may be single-stranded or double-stranded and may represent the sense or the antisense strand, or to any DNA-like or RNA-like material. An “RNA equivalent,” in reference to a DNA sequence, is composed of the same linear sequence of nucleotides as the reference DNA sequence with the exception that all occurrences of the nitrogenous base thymine are replaced with uracil, and the sugar backbone is composed of ribose instead of deoxyribose. RNA may be used in the methods described herein and/or may be converted to cDNA by reverse-transcription for use in the methods described herein.

As used herein, “amplification” or “amplifying” refers to the production of additional copies of a nucleic acid sequence. Amplification is generally carried out using polymerase chain reaction (PCR) technologies known in the art. The term “amplification reaction system” refers to any in vitro means for multiplying the copies of a target sequence of nucleic acid. The term “amplification reaction mixture” refers to an aqueous solution comprising the various reagents used to amplify a target nucleic acid. These may include enzymes (e.g., a thermostable polymerase), aqueous buffers, salts, amplification primers, target nucleic acid, and nucleoside triphosphates, and optionally at least one labeled probe and/or optionally at least one agent for determining the melting temperature of an amplified target nucleic acid (e.g., a fluorescent intercalating agent that exhibits a change in fluorescence in the presence of double-stranded nucleic acid).

The amplification methods described herein my include “real-time monitoring” or “continuous monitoring.” These terms refer to monitoring multiple times during a cycle of PCR, preferably during temperature transitions, and more preferably obtaining at least one data point in each temperature transition. The term “homogeneous detection assay” is used to describe an assay that includes coupled amplification and detection, which may include “real-time monitoring” or “continuous monitoring.”

Amplification of nucleic acids may include amplification of nucleic acids or subregions of these nucleic acids. For example, amplification may include amplifying portions of nucleic acids between 100 and 300 bases long by selecting the proper primer sequences and using the PCR.

The disclosed methods may include amplifying at least one or more nucleic acids in the sample. In the disclosed methods, amplification may be monitored using real-time methods.

Amplification mixtures may include natural nucleotides (including A, C, G, T, and U) and non-natural nucleotides (e.g., including iC and iG). DNA and RNA are oligonucleotides that include deoxyriboses or riboses, respectively, coupled by phosphodiester bonds. Each deoxyribose or ribose includes a base coupled to a sugar. The bases incorporated in naturally-occurring DNA and RNA are adenosine (A), guanosine (G), thymidine (T), cytosine (C), and uridine (U). These five bases are “natural bases”. According to the rules of base pairing elaborated by Watson and Crick, the natural bases can hybridize to form purine-pyrimidine base pairs, where G pairs with C and A pairs with T or U. These pairing rules facilitate specific hybridization of an oligonucleotide with a complementary oligonucleotide.

The formation of these base pairs by the natural bases is facilitated by the generation of two or three hydrogen bonds between the two bases of each base pair. Each of the bases includes two or three hydrogen bond donor(s) and hydrogen bond acceptor(s). The hydrogen bonds of the base pair are each formed by the interaction of at least one hydrogen bond donor on one base with a hydrogen bond acceptor on the other base. Hydrogen bond donors include, for example, heteroatoms (e.g., oxygen or nitrogen) that have at least one attached hydrogen. Hydrogen bond acceptors include, for example, heteroatoms (e.g., oxygen or nitrogen) that have a lone pair of electrons.

The natural or non-natural bases used herein can be derivatized by substitution at non-hydrogen bonding sites to form modified natural or non-natural bases. For example, a base can be derivatized for attachment to a support by coupling a reactive functional group (for example, thiol, hydrazine, alcohol, amine, and the like) to a non-hydrogen bonding atom of the base. Other possible substituents include, for example, biotin, digoxigenin, fluorescent groups, alkyl groups (e.g., methyl or ethyl), and the like.

Non-natural bases, which form hydrogen-bonding base pairs, can be constructed as described, for example, in U.S. Pat. Nos. 5,432,272; 5,965,364; 6,001,983; 6,037,120; U.S. published Application No. 2002/0150900; and U.S. Pat. No. 6,140,496, all of which are incorporated herein by reference. Non-natural nucleosides and non-natural nucleotides may include non-natural bases as disclosed herein. Suitable non-natural bases and their corresponding base pairs may include the following bases in base pair combinations (iso-C/iso-G, K/X, H/J, and M/N): 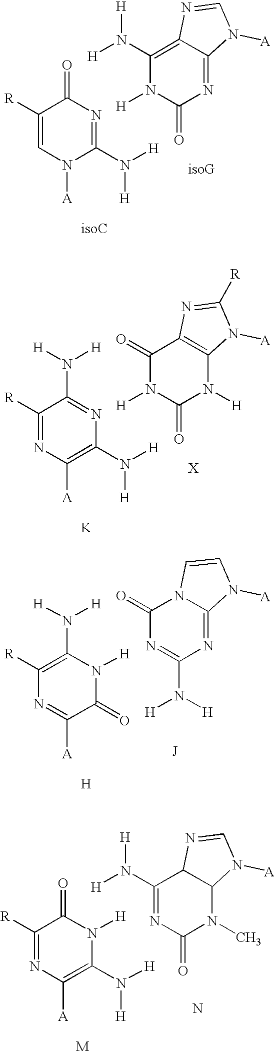
where A is the point of attachment to the sugar or other portion of the polymeric backbone and R is H or a substituted or unsubstituted alkyl group. It will be recognized that other non-natural bases utilizing hydrogen bonding can be prepared, as well as modifications of the above-identified non-natural bases by incorporation of functional groups at the non-hydrogen bonding atoms of the bases.

The hydrogen bonding of these non-natural base pairs is similar to those of the natural bases where two or three hydrogen bonds are formed between hydrogen bond acceptors and hydrogen bond donors of the pairing non-natural bases. One of the differences between the natural bases and these non-natural bases is the number and position of hydrogen bond acceptors and hydrogen bond donors. For example, cytosine can be considered a donor/acceptor/acceptor base with guanine being the complementary acceptor/donor/donor base. Iso-C is an acceptor/acceptor/donor base and iso-G is the complementary donor/donor/acceptor base, as illustrated in U.S. Pat. No. 6,037,120, incorporated herein by reference.

Other non-natural bases for use in oligonucleotides include, for example, naphthalene, phenanthrene, and pyrene derivatives as discussed, for example, in Ren, et al., J. Am. Chem. Soc. 1996, 118:1671 and McMinn et al., J. Am. Chem. Soc. 1999, 121:11585, both of which are incorporated herein by reference. These bases do not utilize hydrogen bonding for stabilization, but instead rely on hydrophobic or van der Waals interactions to form base pairs.

The use of non-natural bases according to the methods disclosed herein is extendable beyond the detection and quantification of nucleic acid sequences present in a sample. For example, non-natural bases can be recognized by many enzymes that catalyze reactions associated with nucleic acids. While a polymerase requires a complementary nucleotide to continue polymerizing an extending oligonucleotide chain, other enzymes do not require a complementary nucleotide. If a non-natural base is present in the template and its complementary non-natural base is not present in the reaction mix, a polymerase will typically stall (or, in some instances, misincorporate a base when given a sufficient amount of time) when attempting to extend an elongating primer past the non-natural base. However, other enzymes that catalyze reactions associated with nucleic acids, such as ligases, kinases, nucleases, polymerases, topoisomerases, helicases, and the like can catalyze reactions involving non-natural bases. Such features of non-natural bases can be taken advantage of, and are within the scope of the presently disclosed methods and kits.

For example, non-natural bases can be used to generate duplexed nucleic acid sequences having a single strand overhang. This can be accomplished by performing a PCR reaction to detect a target nucleic acid in a sample, the target nucleic acid having a first portion and a second portion, where the reaction system includes all four naturally occurring dNTP's, a first primer that is complementary to the first portion of the target nucleic acid, a second primer having a first region and a second region, the first region being complementary to the first portion of the target nucleic acid, and the second region being noncomplementary to the target nucleic acid. The second region of the second primer comprises a non-natural base. The first primer and the first region of the second primer hybridize to the target nucleic acid, if present. Several rounds of PCR will produce an amplification product containing (i) a double-stranded region and (ii) a single-stranded region. The double-stranded region is formed through extension of the first and second primers during PCR. The single-stranded region includes the one or more non-natural bases. The single-stranded region of the amplification product results because the polymerase is not able to form an extension product by polymerization beyond the non-natural base in the absence of the nucleotide triphosphate of the complementary non-natural base. In this way, the non-natural base functions to maintain a single-stranded region of the amplification product.

A polymerase can, in some instances, misincorporate a base opposite a non-natural base. In some embodiments, the misincorporation takes place because the reaction mix does not include a complementary non-natural base. Therefore, if given sufficient amount of time, the polymerase can, in some cases, misincorporate a base that is present in the reaction mixture opposite the non-natural base.

The nucleotides disclosed herein, which may include non-natural nucleotides, may be coupled to a label (e.g., a quencher or a fluorophore). Coupling may be performed using methods known in the art.

The oligonucleotides of the present methods may function as primers. In some embodiments, the oligonucleotides are labeled. For example, the oligonucleotides may be labeled with a reporter that emits a detectable signal (e.g., a fluorophore). The oligonucleotides may include at least one non-natural nucleotide. For example, the oligonucleotides may include at least one nucleotide having a base that is not A, C, G, T, or U (e.g., iC or iG). Where the oligonucleotide is used as a primer for PCR, the amplification mixture may include at least one nucleotide that is labeled with a quencher (e.g., Dabcyl). The labeled nucleotide may include at least one non-natural nucleotide. For example, the labeled nucleotide may include at least one nucleotide having a base that is not A, C, G, T, or U (e.g., iC or iG).

In some embodiments, the oligonucleotide may be designed not to form an intramolecular structure such as a hairpin. In other embodiments, the oligonucleotide may be designed to form an intramolecular structure such as a hairpin. For example, the oligonucleotide may be designed to form a hairpin structure that is altered after the oligonucleotide hybridizes to a target nucleic acid, and optionally, after the target nucleic acid is amplified using the oligonucleotide as a primer.

The oligonucleotide may be labeled with a fluorophore that exhibits quenching when incorporated in an amplified product as a primer. In other embodiments, the oligonucleotide may emit a detectable signal after the oligonucleotide is incorporated in an amplified product as a primer (e.g., inherently, or by fluorescence induction or fluorescence dequenching). Such primers are known in the art (e.g., LightCycler primers, Amplifluor® Primers, Scorpion® Primers and Lux™ Primers). The fluorophore used to label the oligonucleotide may emit a signal when intercalated in double-stranded nucleic acid. As such, the fluorophore may emit a signal after the oligonucleotide is used as a primer for amplifying the nucleic acid.

The oligonucleotides that are used in the disclosed methods may be suitable as primers for amplifying at least one nucleic acid in the sample and as probes for detecting at least one nucleic acid in the sample. In some embodiments, the oligonucleotides are labeled with at least one fluorescent dye, which may produce a detectable signal. The fluorescent dye may function as a fluorescence donor for fluorescence resonance energy transfer (FRET). The detectable signal may be quenched when the oligonucleotide is used to amplify a target nucleic acid. For example, the amplification mixture may include nucleotides that are labeled with a quencher for the detectable signal emitted by the fluorophore. Optionally, the oligonucleotides may be labeled with a second fluorescent dye or a quencher dye that may function as a fluorescence acceptor (e.g., for FRET). Where the oligonucleotide is labeled with a first fluorescent dye and a second fluorescent dye, a signal may be detected from the first fluorescent dye, the second fluorescent dye, or both. Signals may be detected at a gradient of temperatures (e.g., in order to determine a melting temperature for an amplicon or a complex that includes a probe hybridized to a target nucleic acid).

The disclosed methods may be performed with any suitable number of oligonucleotides. Where a plurality of oligonucleotides are used (e.g., two or more oligonucleotides), different oligonucleotide may be labeled with different fluorescent dyes capable of producing a detectable signal. In some embodiments, oligonucleotides are labeled with at least one of two different fluorescent dyes. In further embodiments, oligonucleotides are labeled with at least one of three different fluorescent dyes.

In some embodiments, each different fluorescent dye emits a signal that can be distinguished from a signal emitted by any other of the different fluorescent dyes that are used to label the oligonucleotides. For example, the different fluorescent dyes may have wavelength emission maximums all of which differ from each other by at least about 5 nm (preferably by least about 10 nm). In some embodiments, each different fluorescent dye is excited by different wavelength energies. For example, the different fluorescent dyes may have wavelength absorption maximums all of which differ from each other by at least about 5 nm (preferably by at least about 10 nm).

Where a fluorescent dye is used to determine the melting temperature of a nucleic acid in the method, the fluorescent dye may emit a signal that can be distinguished from a signal emitted by any other of the different fluorescent dyes that are used to label the oligonucleotides. For example, the fluorescent dye for determining the melting temperature of a nucleic acid may have a wavelength emission maximum that differs from the wavelength emission maximum of any other fluorescent dye that is used for labeling an oligonucleotide by at least about 5 nm (preferably by least about 10 nm). In some embodiments, the fluorescent dye for determining the melting temperature of a nucleic acid may be excited by different wavelength energy than any other of the different fluorescent dyes that are used to label the oligonucleotides. For example, the fluorescent dye for determining the melting temperature of a nucleic acid may have a wavelength absorption maximum that differs from the wavelength absorption maximum of any fluorescent dye that is used for labeling an oligonucleotide by at least about 5 nm (preferably by least about 10 nm).

The methods may include determining the melting temperature of at least one nucleic acid in a sample (e.g., an amplicon or a nucleic acid complex that includes a probe hybridized to a target nucleic acid), which may be used to identify the nucleic acid. Determining the melting temperature may include exposing an amplicon or a nucleic acid complex to a temperature gradient and observing a detectable signal from a fluorophore. Optionally, where the oligonucleotides of the method are labeled with a first fluorescent dye, determining the melting temperature of the detected nucleic acid may include observing a signal from a second fluorescent dye that is different from the first fluorescent dye. In some embodiments, the second fluorescent dye for determining the melting temperature of the detected nucleic acid is an intercalating agent. Suitable intercalating agents may include, but are not limited to SYBR™ Green 1 dye, SYBR dyes, Pico Green, SYTO dyes, SYTOX dyes, ethidium bromide, ethidium homodimer-1, ethidium homodimer-2, ethidium derivatives, acridine, acridine orange, acridine derivatives, ethidium-acridine heterodimer, ethidium monoazide, propidium iodide, cyanine monomers, 7-aminoactinomycin D, YOYO-1, TOTO-1, YOYO-3, TOTO-3, POPO-1, BOBO-1, POPO-3, BOBO-3, LOLO-1, JOJO-1, cyanine dimers, YO-PRO-1, TO-PRO-1, YO-PRO-3, TO-PRO-3, TO-PRO-5, PO-PRO-1, BO-PRO-1, PO-PRO-3, BO-PRO-3, LO-PRO-1, JO-PRO-1, and mixture thereof. In suitable embodiments, the selected intercalating agent is SYBR™ Green 1 dye.

Typically, an intercalating agent used in the method will exhibit a change in fluorescence when intercalated in double-stranded nucleic acid. A change in fluorescence may include an increase in fluorescence intensity or a decrease in fluorescence intensity. For example, the intercalating agent may exhibit an increase in fluorescence when intercalated in double-stranded nucleic acid, and a decrease in fluorescence when the double-stranded nucleic acid is melted. A change in fluorescence may include a shift in fluorescence spectra (i.e., a shift to the left or a shift to the right in maximum absorbance wavelength or maximum emission wavelength). For example, the intercalating agent may emit a fluorescent signal of a first wavelength (e.g., green) when intercalated in double-stranded nucleic and emit a fluorescent signal of a second wavelength (e.g., red) when not intercalated in double-stranded nucleic acid. A change in fluorescence of an intercalating agent may be monitored at a gradient of temperatures to determine the melting temperature of the nucleic acid (where the intercalating agent exhibits a change in fluorescence when the nucleic acid melts).

In the disclosed methods, each of the amplified target nucleic acids may have different melting temperatures. For example, each of the amplified target nucleic acids may have a melting temperature that differs by at least about 1° C., more preferably by at least about 2° C., or even more preferably by at least about 4° C. from the melting temperature of any of the other amplified target nucleic acids.

The methods disclosed herein may include transcription of RNA to DNA (i.e., reverse transcription). For example, reverse transcription may be performed prior to amplification.

As used herein, “labels” or “reporter molecules” are chemical or biochemical moieties useful for labeling a nucleic acid, amino acid, or antibody. “Labels” and “reporter molecules” include fluorescent agents, chemiluminescent agents, chromogenic agents, quenching agents, radionuclides, enzymes, substrates, cofactors, inhibitors, scintillation agents, magnetic particles, and other moieties known in the art. “Labels” or “reporter molecules” are capable of generating a measurable signal and may be covalently or noncovalently joined to an oligonucleotide.

As used herein, a “fluorescent dye” or a “fluorophore” is a chemical group that can be excited by light to emit fluorescence. Some suitable fluorophores may be excited by light to emit phosphorescence. Dyes may include acceptor dyes that are capable of quenching a fluorescent signal from a fluorescent donor dye. Dyes that may be used in the disclosed methods include, but are not limited to, the following dyes and/or dyes sold under the following tradenames: 1,5 IAEDANS; 1,8-ANS; 4-Methylumbelliferone; 5-carboxy-2,7-dichlorofluorescein; 5-Carboxyfluorescein (5-FAM); 5-Carboxytetramethylrhodamine (5-TAMRA); 5-FAM (5-Carboxyfluorescein); 5-HAT (Hydroxy Tryptamine); 5-Hydroxy Tryptamine (HAT); 5-ROX (carboxy-X-rhodamine); 5-TAMRA (5-Carboxytetramethylrhodamine); 6-Carboxyrhodamine 6G; 6-CR 6G; 6-JOE; 7-Amino-4-methylcoumarin; 7-Aminoactinomycin D (7-AAD); 7-Hydroxy-4-methylcoumarin; 9-Amino-6-chloro-2-methoxyacridine; ABQ; Acid Fuchsin; ACMA (9-Amino-6-chloro-2-methoxyacridine); Acridine Orange; Acridine Red; Acridine Yellow; Acriflavin; Acriflavin Feulgen SITSA; Alexa Fluor 350™; Alexa Fluor 430™; Alexa Fluor 488™; Alexa Fluor 532™; Alexa Fluor 546™; Alexa Fluor 568™; Alexa Fluor 594™; Alexa Fluor 633™; Alexa Fluor 647™; Alexa Fluor 660™; Alexa Fluor 680™; Alizarin Complexon; Alizarin Red; Allophycocyanin (APC); AMC; AMCA-S; AMCA (Aminomethylcoumarin); AMCA-X; Aminoactinomycin D; Aminocoumarin; Aminomethylcoumarin (AMCA); Anilin Blue; Anthrocyl stearate; APC (Allophycocyanin); APC-Cy7; APTS; Astrazon Brilliant Red 4G; Astrazon Orange R; Astrazon Red 6B; Astrazon Yellow 7 GLL; Atabrine; ATTO-TAG™ CBQCA; ATTO-TAG™ FQ; Auramine; Aurophosphine G; Aurophosphine; BAO 9 (Bisaminophenyloxadiazole); Berberine Sulphate; Beta Lactamase; BFP blue shifted GFP (Y66H); Blue Fluorescent Protein; BFP/GFP FRET; Bimane; Bisbenzamide; Bisbenzimide (Hoechst); Blancophor FFG; Blancophor SV; BOBO™-1; BOBO™-3; Bodipy 492/515; Bodipy 493/503; Bodipy 500/510; Bodipy 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy FL; Bodipy FL ATP; Bodipy F1-Ceramide; Bodipy R6G SE; Bodipy TMR; Bodipy TMR-X conjugate; Bodipy TMR-X, SE; Bodipy TR; Bodipy TR ATP; Bodipy TR-X SE; BO-PRO™-1; BO-PRO™-3; Brilliant Sulphoflavin FF; Calcein; Calcein Blue; Calcium Crimson™; Calcium Green; Calcium Orange; Calcofluor White; Carboxy-X-rhodamine (5-ROX); Cascade Blue™; Cascade Yellow; Catecholamine; CCF2 (GeneBlazer); CFDA; CFP-Cyan Fluorescent Protein; CFP/YFP FRET; Chlorophyll; Chromomycin A; CL-NERF (Ratio Dye, pH); CMFDA; Coelenterazine f; Coelenterazine fcp; Coelenterazine h; Coelenterazine hcp; Coelenterazine ip; Coelenterazine n; Coelenterazine 0; Coumarin Phalloidin; C-phycocyanine; CPM Methylcoumarin; CTC; CTC Formazan; Cy2™; Cy3.1 8; Cy3.5™; Cy3™; Cy5.1 8; Cy5.5™; Cy5™; Cy7™; Cyan GFP; cyclic AMP Fluorosensor (FiCRhR); Dabcyl; Dansyl; Dansyl Amine; Dansyl Cadaverine; Dansyl Chloride; Dansyl DHPE; Dansyl fluoride; DAPI; Dapoxyl; Dapoxyl 2; Dapoxyl 3; DCFDA; DCFH (Dichlorodihydrofluorescein Diacetate); DDAO; DHR (Dihydorhodamine 123); Di-4-ANEPPS; Di-8-ANEPPS (non-ratio); DiA (4-Di-16-ASP); Dichlorodihydrofluorescein Diacetate (DCFH); DiD-Lipophilic Tracer; DiD (DiIC18(5)); DIDS; Dihydorhodamine 123 (DHR); DiI (DiC18(3)); Dinitrophenol; DiO (DiOC18(3)); DiR; DiR (DiIC18(7)); DNP; Dopamine; DsRed; DTAF; DY-630-NHS; DY-635-NHS; EBFP; ECFP; EGFP; ELF 97; Eosin; Erythrosin; Erythrosin ITC; Ethidium Bromide; Ethidium homodimer-1 (EthD-1); Euchrysin; EukoLight; Europium (III) chloride; EYFP; Fast Blue; FDA; Feulgen (Pararosaniline); FITC; Flazo Orange; Fluo-3; Fluo-4; Fluorescein (FITC); Fluorescein Diacetate; Fluoro-Emerald; Fluoro-Gold (Hydroxystilbamidine); Fluor-Ruby; Fluor X; FM 1-43™; FM 4-46; Fura Red™; Fura Red™/Fluo-3; Fura-2; Fura-2/BCECF; Genacryl Brilliant Red B; Genacryl Brilliant Yellow IOGF; Genacryl Pink 3G; Genacryl Yellow 5GF; GeneBlazer (CCF2); GFP (S65T); GFP red shifted (rsGFP); GFP wild type, non-UV excitation (wtGFP); GFP wild type, UV excitation (wtGFP); GFPuv; Gloxalic Acid; Granular Blue; Haematoporphyrin; Hoechst 33258; Hoechst 33342; Hoechst 34580; HPTS; Hydroxycoumarin; Hydroxystilbamidine (FluoroGold); Hydroxytryptamine; Indo-1; Indodicarbocyanine (DiD); Indotricarbocyanine (DiR); Intrawhite Cf; JC-1; JO-JO-1; JO-PRO-1; Laurodan; LDS 751 (DNA); LDS 751 (RNA); Leucophor PAF; Leucophor SF; Leucophor WS; Lissamine Rhodamine; Lissamine Rhodamine B; Calcein/Ethidium homodimer; LOLO-1; LO-PRO-1; Lucifer Yellow; Lyso Tracker Blue; Lyso Tracker Blue-White; Lyso Tracker Green; Lyso Tracker Red; Lyso Tracker Yellow; LysoSensor Blue; LysoSensor Green; LysoSensor Yellow/Blue; Mag Green; Magdala Red (Phloxin B); Mag-Fura Red; Mag-Fura-2; Mag-Fura-5; Mag-Indo-1; Magnesium Green; Magnesium Orange; Malachite Green; Marina Blue; Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin; Monobromobimane; Monobromobimane (mBBr-GSH); Monochlorobimane; MPS (Methyl Green Pyronine Stilbene); NBD; NBD Amine; Nile Red; NED™; Nitrobenzoxadidole; Noradrenaline; Nuclear Fast Red; Nuclear Yellow; Nylosan Brilliant Iavin E8G; Oregon Green; Oregon Green 488-X; Oregon Green™; Oregon Green™ 488; Oregon Green™ 500; Oregon Green™ 514; Pacific Blue; Pararosaniline (Feulgen); PBFI; PE-Cy5; PE-Cy7; PerCP; PerCP-Cy5.5; PE-TexasRed [Red 613]; Phloxin B (Magdala Red); Phorwite AR; Phorwite BKL; Phorwite Rev; Phorwite RPA; Phosphine 3R; Phycoerythrin B [PE]; Phycoerythrin R [PE]; PKH26 (Sigma); PKH67; PMIA; Pontochrome Blue Black; POPO-1; POPO-3; PO-PRO-1; PO-PRO-3; Primuline; Procion Yellow; Propidium Iodid (PI); PyMPO; Pyrene; Pyronine; Pyronine B; Pyrozal Brilliant Flavin 7GF; QSY 7; Quinacrine Mustard; Red 613 [PE-TexasRed]; Resorufin; RH 414; Rhod-2; Rhodamine; Rhodamine 110; Rhodamine 123; Rhodamine 5 GLD; Rhodamine 6G; Rhodamine B; Rhodamine B 200; Rhodamine B extra; Rhodamine BB; Rhodamine BG; Rhodamine Green; Rhodamine Phallicidine; Rhodamine Phalloidine; Rhodamine Red; Rhodamine WT; Rose Bengal; R-phycocyanine; R-phycoerythrin (PE); RsGFP; S65A; S65C; S65L; S65T; Sapphire GFP; SBFI; Serotonin; Sevron Brilliant Red 2B; Sevron Brilliant Red 4G; Sevron Brilliant Red B; Sevron Orange; Sevron Yellow L; sgBFP™; sgBFP™ (super glow BFP); sgGFP™; sgGFP™ (super glow GFP); SITS; SITS (Primuline); SITS (Stilbene Isothiosulphonic Acid); SNAFL calcein; SNAFL-1; SNAFL-2; SNARF calcein; SNARF1; Sodium Green; SpectrumAqua; SpectrumGreen; SpectrumOrange; Spectrum Red; SPQ (6-methoxy-N-(3-sulfopropyl)quinolinium); Stilbene; Sulphorhodamine B can C; Sulphorhodamine G Extra; SYTO 11; SYTO 12; SYTO 13; SYTO 14; SYTO 15; SYTO 16; SYTO 17; SYTO 18; SYTO 20; SYTO 21; SYTO 22; SYTO 23; SYTO 24; SYTO 25; SYTO 40; SYTO 41; SYTO 42; SYTO 43; SYTO 44; SYTO 45; SYTO 59; SYTO 60; SYTO 61; SYTO 62; SYTO 63; SYTO 64; SYTO 80; SYTO 81; SYTO 82; SYTO 83; SYTO 84; SYTO 85; SYTOX Blue; SYTOX Green; SYTOX Orange; TET™; Tetracycline; Tetramethylrhodamine (TRITC); Texas Red™; Texas Red-X™ conjugate; Thiadicarbocyanine (DiSC3); Thiazine Red R; Thiazole Orange; Thioflavin 5; Thioflavin S; Thioflavin TCN; Thiolyte; Thiozole Orange; Tinopol CBS (Calcofluor White); TMR; TO-PRO-1; TO-PRO-3; TO-PRO-5; TOTO-1; TOTO-3; TriColor (PE-Cy5); TRITC TetramethylRodaminelsoThioCyanate; True Blue; TruRed; Ultralite; Uranine B; Uvitex SFC; VIC®; wt GFP; WW 781; X-Rhodamine; XRITC; Xylene Orange; Y66F; Y66H; Y66W; Yellow GFP; YFP; YO-PRO-1; YO-PRO-3; YOYO-1; YOYO-3; and salts thereof.

Fluorescent dyes or fluorophores may include derivatives that have been modified to facilitate conjugation to another reactive molecule. As such, fluorescent dyes or fluorophores may include amine-reactive derivatives such as isothiocyanate derivatives and/or succinimidyl ester derivatives of the fluorophore.

The oligonucleotides and nucleotides of the disclosed methods may be labeled with a quencher. Quenching may include dynamic quenching (e.g., by FRET), static quenching, or both. Suitable quenchers may include Dabcyl. Suitable quenchers may also include dark quenchers, which may include black hole quenchers sold under the tradename “BHQ” (e.g., BHQ-0, BHQ-1, BHQ-2, and BHQ-3, Biosearch Technologies, Novato, Calif.). Dark quenchers also may include quenchers sold under the tradename “QXL™” (Anaspec, San Jose, Calif.). Dark quenchers also may include DNP-type non-fluorophores that include a 2,4-dinitrophenyl group.

The oligonucleotide of the present methods may be labeled with a donor fluorophore and an acceptor fluorophore (or quencher dye) that are present in the oligonucleotides at positions that are suitable to permit FRET (or quenching). Labeled oligonucleotides that are suitable for the present methods may include but are not limited to oligonucleotides designed to function as LightCycler primers or probes, Taqman® Probes, Molecular Beacon Probes, Amplifluor® Primers, Scorpion® Primers, and LuX™ Primers.

In some embodiments, the detection of coronaviruses that are capable of causing SARS or SARS-like symptoms may be performed using MultiCode®-RTx PCR technology, which is disclosed in U.S. Pat. No. 6,977,161, Patent Application Publication No. 2002-0150900 and WO/01/90417, incorporated herein by reference in their entireties. The assays may be performed using real-time or continuous methods using any suitable commercial thermal cycler. The disclosed technology may be used to detect nucleic acid targets obtained from any source (e.g., human, animal and infectious disease samples).

### ILLUSTRATIVE EMBODIMENTS

The following illustrative embodiments are presented to aid the reader in understanding the methods and kits described herein and are not intended to be limiting.

A first illustrative embodiment includes a method for identifying a SARS virus in a sample comprising: (a) reacting a reaction mixture, the mixture comprising: (i) the sample; (ii) at least one oligonucleotide comprising at least one non-natural base, wherein the oligonucleotide is capable of specifically hybridizing to SARS virus nucleic acid; and (b) detecting the SARS virus nucleic acid if present in the sample.

A second illustrative embodiments includes the method of illustrative embodiment one, wherein the SARS virus is the SARS-associated coronavirus encoded by Genbank Accession No. NC_{—}004718.

A third illustrative embodiment includes the method of illustrative embodiment one, wherein the reaction mixture further comprises: (iii) internal control nucleic acid; and (iv) at least one oligonucleotide capable of specifically hybridizing to the internal control nucleic acid; and the method further comprises: (c) detecting the internal control nucleic acid.

A fourth illustrative embodiment includes the method of illustrative embodiment one wherein the at least one oligonucleotide is capable of specifically hybridizing to a SARS nucleic acid sequence selected from the group consisting of SARS virus nucleoprotein nucleic acid sequence, SARS virus polymerase nucleic acid sequence, SARS virus P65 nucleic acid sequence, and a combination thereof.

A fifth illustrative embodiment includes the method of illustrative embodiment one, wherein the at least one oligonucleotide comprises a nucleic acid sequence selected from the group consisting of SEQ ID NOs: 4-9.

A sixth illustrative embodiment includes the method of illustrative embodiment one, further comprising performing reverse transcription of SARS virus RNA.

A seventh illustrative embodiment includes the method of illustrative embodiment one, further comprising performing RT-PCR of SARS virus RNA.

An eighth illustrative embodiment includes the method of illustrative embodiment seven, further comprising determining a melting temperature of the amplified SARS nucleic acid.

A ninth illustrative embodiment includes the method of illustrative embodiment one, wherein the reaction mixture comprises at least two oligonucleotides capable of specifically hybridizing to SARS virus nucleic acid and the method further comprises amplifying SARS virus nucleic acid using the two oligonucleotides as primers.

A tenth illustrative embodiment includes the method of illustrative embodiment nine, wherein at least one of the two oligonucleotides used as primers includes a label.

An eleventh illustrative embodiment includes the method of illustrative embodiment ten, wherein at least one of the two oligonucleotides used as primers includes at least one base other than A, C, G, T, and U.

A twelfth illustrative embodiment includes the method of illustrative embodiment eleven, wherein the base other than A, C, G, T, and U, is selected from iC and iG.

A thirteenth illustrative embodiment includes the method of illustrative embodiment twelve, wherein the label comprises a fluorophore and the reaction mixture further comprises a nucleotide covalently linked to a quencher.

A fourteenth illustrative embodiment includes the method of illustrative embodiment thirteen, wherein the nucleotide covalently linked to the quencher comprises iC or iG.

A fifteenth illustrative embodiment includes the method of illustrative embodiment three, wherein the internal control nucleic acid comprises a nucleic acid sequence selected from the group consisting of SEQ ID NOs: 15 and 16.

A sixteenth illustrative embodiment includes the method of illustrative embodiment one, wherein the method detects SARS virus nucleic acid that is present in the sample as no more than about 10 copies.

A seventeenth illustrative embodiment includes a method for detecting SARS virus in a sample comprising: (a) reacting a mixture that comprises: (i) the sample; (ii) a first pair of oligonucleotides capable of specifically hybridizing to SARS virus nucleic acid, wherein at least one oligonucleotide of the first pair includes a first label; (iii) control nucleic acid; and (iv) a second pair of oligonucleotides capable of specifically hybridizing to the control nucleic acid, wherein at least one oligonucleotide of the second pair includes a second label, and the first label and second label are different; (b) amplifying and detecting the control nucleic acid and the SARS virus nucleic acid, if present in the sample.

An eighteenth illustrative embodiment includes the method of illustrative embodiment seventeen, wherein the SARS virus is the SARS-associated CoV encoded by the nucleic acid sequence deposited as Genbank Accession No. NC_{—}004718.

A nineteenth illustrative embodiment includes the method of illustrative embodiment seventeen, wherein the first pair of oligonucleotides is capable of specifically hybridizing to nucleic acid selected from the group consisting of nucleic acid of SARS virus polymerase, nucleic acid of SARS virus nucleoprotein, nucleic acid of SARS virus P65 nucleic acid sequence, and combinations thereof.

A twentieth illustrative embodiment includes the method of illustrative embodiment seventeen, wherein at least one oligonucleotide of the first pair of oligonucleotides includes at least one base other than A, C, G, T, and U.

A twenty-first illustrative embodiment includes the method of illustrative embodiment twenty, wherein the base other than A, C, G, T, and U, is selected from iC and iG.

A twenty-second illustrative embodiment includes the method of illustrative embodiment seventeen, wherein at least one oligonucleotide of the second pair of oligonucleotides includes at least one base other than A, C, G, T, and U.

A twenty-third illustrative embodiment includes the method of illustrative embodiment twenty-two, wherein the base other than A, C, G, T, and U, is selected from iC and iG.

A twenty-fourth illustrative embodiment includes the method of illustrative embodiment seventeen, wherein the first label and second label comprise two different fluorophores and the reaction mixture further comprises a nucleotide covalently linked to a quencher that is capable of quenching the two different fluorophores.

A twenty-fifth illustrative embodiment includes the method of illustrative embodiment seventeen, further comprising determining a melting temperature of at least one of amplified nucleic acid of SAR virus nucleic acid and amplified control nucleic acid.

A twenty-sixth illustrative embodiment includes the method of illustrative embodiment seventeen, wherein the method detects SARS virus nucleic that is present in the sample as no more than about 10 copies.

A twenty-seventh illustrative embodiment includes a kit comprising: (a) a first pair of oligonucleotides capable of specifically hybridizing to a SARS virus nucleic acid, wherein at least one oligonucleotide of the first pair comprises at least one non-natural base and a label.

A twenty-eighth illustrative embodiment includes the kit of illustrative embodiment twenty-seven, further comprising: (b) control nucleic acid; and (c) a second pair of oligonucleotides capable of specifically hybridizing to the control nucleic acid, wherein at least one oligonucleotide of the second pair comprises at least one non-natural base and a second label; wherein the first label and second label are different.

A twenty-ninth illustrative embodiment includes the kit of illustrative embodiment twenty-seven, further comprising: (d) a nucleotide that includes a non-natural base that base-pairs with the non-natural base present in the at least one oligonucleotide of the first pair and the non-natural base present in the at least one oligonucleotide of the second pair.

### EXAMPLES

The diversity and utility of the methods are demonstrated in the following examples which are meant to be instructive and not limiting.

1. Primers

Primer and control oligonucleotide designations and sequences used in the examples can be found in Table 2. Oligonucletides used in the assays described herein were designed based on the SARS-CoV sequence deposited in GenBank, Accession No. NC_{—}004718. Primer design packages that may be used for the methods include Primer Express (Applied Biosystems, Foster City, Calif.), Primer3 (see e.g., Krawetz S, Misener S, Bioinformatics Methods and Protocols: Methods in Molecular Biology, Humana Press, Totowa N.J., pp 365-386; Rozen, et al., Methods Mol. Biol. 2000, 132: 365-86) and Visual OMP (DNA Software, Inc., Ann Arbor, Mich.). Here, Primer3 and Visual OMP were used. Incorporation of the iC (X) containing nucleotides during synthesis was done using standard coupling conditions. All synthetic DNAs were quantitated by using extinction coefficients corresponding to the nucleotide makeup and examining initial stocks by OD 260. The DNAs were diluted to appropriate working concentrations in 10 mM MOPS and 0.1 mM EDTA. BLASTN searches were performed for all primers and probes to eliminate priming to sequences other than those specified.

2. Target Selection Criteria and Primers

Targets are selected using BLAST analysis of the SARS-CoV nucleic acid of interest, such as, for example the Genbank Accession No. NC_{—}004718. A non-complementary region from bp 1-3150 is selected and primers are designed. Three sets of primers are selected and tested in a duplex assay with an internal control system that includes an internal control target and an internal control target primer set. A system is designed such that few or no primer dimers are observed after 50 cycles of PCR.

3. Samples

Samples may be prepared by methods known in the art. Exemplary commercially available kits include Qiagen Products, for example QiaAmp RNA Blood MiniKit (Cat.# 52304, Qiagen, Valencia, Calif.). Those skilled in the art will understand that any of a number of different methods of sample preparation will be appropriate for the present methods.

4. Additional Components

In addition to primers and control sequences (listed in Table 2) and target samples, the following exemplary components, known in the art or commercially available, may be used in the described methods.

Reverse Transcriptase: A suitable reverse transcriptase is M-MLV reverse transcriptase. For example, M-MLV provided by Promega Corp. (cat.# M1701, Madison, Wis.) at 25U/μL, 50 μL volume; final concentration 0.5 U/μL (100 reactions).

DNA Polymerase: A suitable DNA polymerase is Titanium Taq Polymerase (100 μl) (Clontech cat# 8434-1, Carlsbad, Calif.) 50× stock concentration, 1× final concentration (200 reactions).

Nuclease Free Water.

Dithiothreiotol (DTT): (e.g., 250 mM stock solution is used in the following examples).

MgCl₂: (e.g., 25 mM stock solution is used in the following examples).

Reaction Buffer: Stock solutions which yield 1× reaction conditions as described in Table 5 may be used to simplify reaction set-up (e.g., 2× ISOlution Buffer, EraGen, Madison, Wis.) for P65, POL and NP detection (Eragen, Madison, Wis.).

5. Assay Setup

For each sample to be run, the total reaction mix may be formulated according to Table 3 (POL and NP) and Table 4 (P65). Total Reaction Size: 25 μL (20 μL Reaction Mix, 5 μL Target). Working concentration (1×) of components in an exemplary PCR reaction for individual 20 μl PCR reaction volumes is shown in Table 5.

6. Reaction Procedure

Reaction mixtures are prepared on ice. Components are thawed and full resuspension of 2× Reaction Buffer is confirmed. Gentle warming by hand is performed if precipitate remains in 2× Reaction Buffer after thawing. Thawed reagents are vortexed.

Reaction mixtures are prepared by mixing appropriate volumes of 2× Reaction Buffer, MgCl₂, DTT, and Nuclease Free Water (see e.g., Tables 2 and 3). Titanium Taq is added to the mixtures. The mixtures are vortexed and incubated on ice for an additional minute. Fifty× (50×) Primer Mix and Internal Control DNA are added and the mixtures are vortexed thoroughly. Internal control RNA is added to all reaction mixtures. Twenty microliters (20 μL) of reaction mix are added to each reaction tube. Five microliters (5 μL) of Dilution Buffer is added to “no target” sample wells or 5 μL of target is added to sample wells. Reaction tubes or plates are spun at ˜2000 rpm. Tubes are inserted into instrument and run.

Thermocycling Parameters:

Exemplary conditions for PCR for p65, Pol and NP are as follows.

Stage 1
50° C./300 seconds (for reactions including M-MuLV RT)

Stage 2
95° C./120 Seconds

Stage 3
95° C./5 Seconds
55° C./5 Seconds
74° C./20 Seconds (Optical Reading)
(Repeat Stage 3 45 times for P65, repeat 50 times for POL and NP)

Stage 4
60° C./15 Seconds

Stage 5
Start Temp=60° C.
End Temp=95° C.
Increment=0.2° C./Second
   Upon completion of stages 1-5, a channel 4 melt should be set up as follows:

Stage 1
60° C./15 seconds

Stage 2
Start Temp=60° C.
End Temp=95° C.
Increment=0.2° C./Second
   7. Analysis Software

Commercially-available real-time thermal cyclers use software designed to analyze reactions where fluorescence increases with PCR product accumulation. To analyze decreasing fluorescence results, analysis software was developed that imports RTx raw data and performs cycle threshold and melt curve analyses. Raw F1 and F2 component fluorescence data for both amplification and melt programs were exported from the Light Cycler-1 Analysis software (Version 5.32) as text files and analyzed with EraGen Real-time Run Importer and Analysis Desktop v0.9.8 alpha (EraGen Biosciences, Inc., Madison, Wis.).

8. Reactions with Polymerase (POL) and Nucleocapsid (NP) Targets

The PCR primers specific for POL and NP sequences (SEQ ID NOs 4, 5 and 6, 7 respectively) were designed to achieve a predicted Tm of 60° C. The reverse primer used to transcribe the SARS-CoV RNA sequence into cDNA included all standard deoxyoligonulceotides (SEQ ID NOs. 5 and 7) while the forward primer contained a single 5′ 5-methyl deoxyisocytosine (iC) adjacent to a terminal FAM fluorophore (SEQ ID NOs. 4 and 6).

An RNA internal control is included in the reaction mix at a final concentration of 1×10⁴ copies per reaction. For example, internal control RNA template SEQ ID NO. 15 was used with internal control primers SEQ ID NOs. 10 and 11. One member of the control primer pair includes a label different than the SARS virus primer pair. Here, for example, control primer SEQ ID NO. 10 is HEX-labeled, while the SARS forward primer (SEQ ID NO. 4 and 6) are FAM labeled.

Five microliters of target nucleic acid were added to each reaction vessel. Target sample nucleic acid was added at zero to 1×10⁷ copies per reaction as estimated by absorbance at 260 nm. DNA oligonucleotide containing SARS target sequence was used as positive control (SEQ ID NO. 12 POL sequence, or SEQ ID NO. 13 NP sequence) at a concentration of 1×10⁴ copies per reaction.

PCR and RT-PCR reactions were performed. For RNA templates, M-MuLV RT (Promega, Madison, Wis.) was added at 0.5 units/μl, and an initial 5 minute incubation at 50° C. was performed prior to PCR amplification to reverse transcribe RNA to DNA. PCR conditions were as follows. 1× Reaction Buffer (e.g., ISOlution Buffer, EraGen, Madison, Wis.); 300 nM PCR primers; 5 mM dithiothreitol; 2 mM MgCl₂ (NP) or 4 mM MgCl₂ (POL); Titanium Taq DNA polymerase (Clontech, CA) at manufacturer's recommended concentration. PCR cycling parameters were 2 minute denature at 95° C. followed by 50 cycles of 5 seconds at 95° C., 5 seconds at 55° C., 20 seconds at 72° C., with optical read on the Prism 7700 (Applied Biosystems Inc., Foster City, Calif.) real-time thermal cycler. A thermal melt with optical read from 60° to 95° C. was performed directly following the last 72° C. step of thermal cycling. Raw FAM component fluorescence data was exported from SDS 1.9 (Applied Biosystems, Inc.) software and analyzed with GeneCode software (EraGen, Madison, Wis.).

9. Reactions with P65 Target

The PCR primers specific for P65 sequences (SEQ ID NOs 8 and 9) were also designed to achieve a predicted Tm of 60° C. The reverse primer used to transcribe the SARS-CoV RNA sequence into cDNA included all standard deoxyoligonulceotides (SEQ ID NO. 9) while the forward primer contained a single 5′ 5-methyl deoxyisocytosine (iC) adjacent to a terminal FAM fluorophore (SEQ ID NO. 8). An RNA internal control was also included in the reaction mix. For example, internal control RNA template SEQ ID NO. 16 may be used with internal control primers SEQ ID NOs 10 and 11. One member of the control primer pair is labeled differently then the labeled member of the target primer pair. Here, for example, control primer SEQ ID NO. 10 may be HEX or ROX labeled; different labels may be used for different experiments. In some embodiments, HEX may be preferred. Additionally, an RNA control template including SARS-CoV P65 sequences, such as SEQ ID NO: 14, may also be run as a separate reaction control.

RT and PCR conditions were similar for the P65 targets. RT-PCR reactions were performed using from zero to 1×10⁷ copies of DNA and RNA targets as estimated by absorbance at 260 nm. For RNA templates, M-MuLV RT (Promega, Madison, Wis.) was added at 0.5 units/μL, and an initial 5 minute incubation at 50° C. was performed prior to PCR amplification to reverse transcribe RNA to DNA. PCR conditions were as follows. 1× Reaction Buffer (e.g., ISOlution Buffer, EraGen, Madison, Wis.); 300 nM PCR primers; 5 mM dithiothreitol; 2 mM MgCl₂, Titanium Taq DNA polymerase (Clontech, CA) at manufacturer's recommended concentration. PCR cycling parameters were as follows: 2 minute denature at 95° C. followed by 45 cycles of 5 seconds at 95° C., 5 seconds at 55° C., 20 seconds at 72° C., with optical read on the Prism 7700 (Applied Biosystems Inc., Foster City, Calif.) real-time thermal cycler. A thermal melt with optical read from 60 to 95° C. at increments of 0.2° C./second was performed directly following the last 72° C. step of thermal cycling. Raw FAM component fluorescence data was exported from SDS 1.9 (Applied Biosystems, Inc.) software and analyzed with GeneCode 2.0 Analysis Software (EraGen, Madison, Wis.).

10. SARS-CoV Isolation from Infected Cells and Detection

SARS-CoV Tor-2 virus was grown in Vero cells for 72 hours in EMEM supplemented with 5% FBS, 1% PenStrep, 1% 1M HEPES, 0.5% Fungizone, and 0.5% gentamicin. CPE at 72 hours was +3 with about 70% cell death.

SARS-CoV Tor-2 passage 4 was grown in confluent Vero E6 cell monolayers in minimal essential medium containing 3% fetal bovine serum. The monolayers were infected at ˜1 PFU/mL, and at 48 hours post infection the cells and supernatant were harvested. Free virus was separated from the infected cell fraction by centrifugation at 3,500 rpm for 10 minutes. Both the free virus and infected cell fractions were then RNA extracted. RNA isolation from cells was carried out using Trizol™ LS (Invitrogen, Carlasbad, Calif.) according to the manufacturer's instructions. Briefly, chloroform was added to the Trizol/virus mixture and phases were separated by centrifugation. The RNA-containing aqueous phase was collected and subsequently precipitated with isopropranol. RNA was collected by centrifugation, washed with 75% ethanol, and resuspended in RNAse free water. RNA was aliquoted, measured by spectrophotometry, and stored at −70° C. until use.

SARS viral titer was ascertained by determining the number of plaque forming units (“PFU”) as follows. PFU determination was performed in a class II biological safety cabinet under BSL3 containment. Ten-fold serial dilutions were prepared in Hanks' Balanced Salt Solution (HBSS) media containing 5% heat-inactivated FBS, 1% Pen-Strep, 0.5% gentamicin, and 0.5% fungizone. For each dilution, two wells of confluent Vero cell monolayers were inoculated with 0.1 ml of virus. Plates were incubated at 37° C. for 1 hour in a CO₂ incubator. Two milliliters (mL) of overlay media (2× EBME containing 5% heat-inactivated FBS, 1% Pen-Strep, 0.5% gentamicin, and 0.5% fungizone, without phenol red) was added. Overlays were allowed to solidify at room temperature, and plates were incubated for 37° C. for 24 hour in a CO₂ incubator. On day two, 2.0 mL of overlay media (2× EBME containing 5% heat-inactivated FBS, 1% Pen-Strep, 0.5% gentamicin, and 0.5% fungizone, with phenol red) was added, overlays were allowed to solidify at room temperature, and plates were incubated at 37° C. for in a CO₂ incubator. Plates were checked daily for plaque formation. Plaques were enumerated and titer endpoints were confirmed by observing at least one dilution containing less than 10 PFU/well for both replicates.

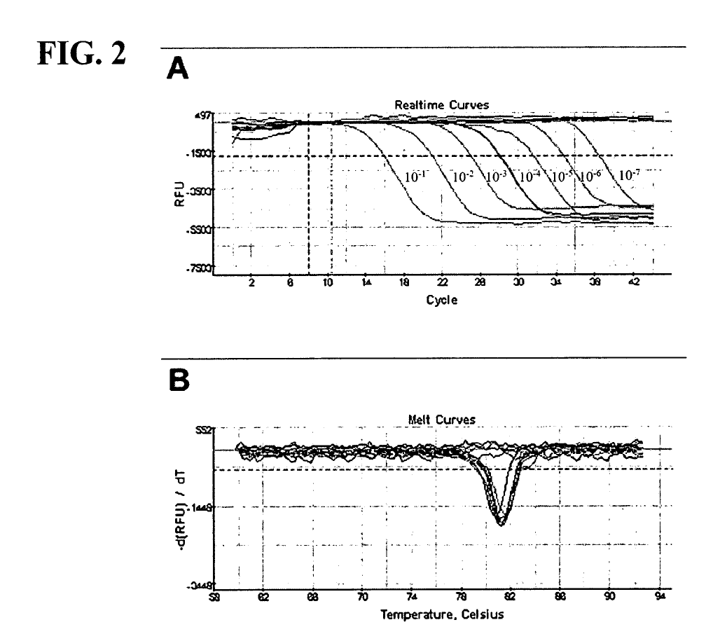

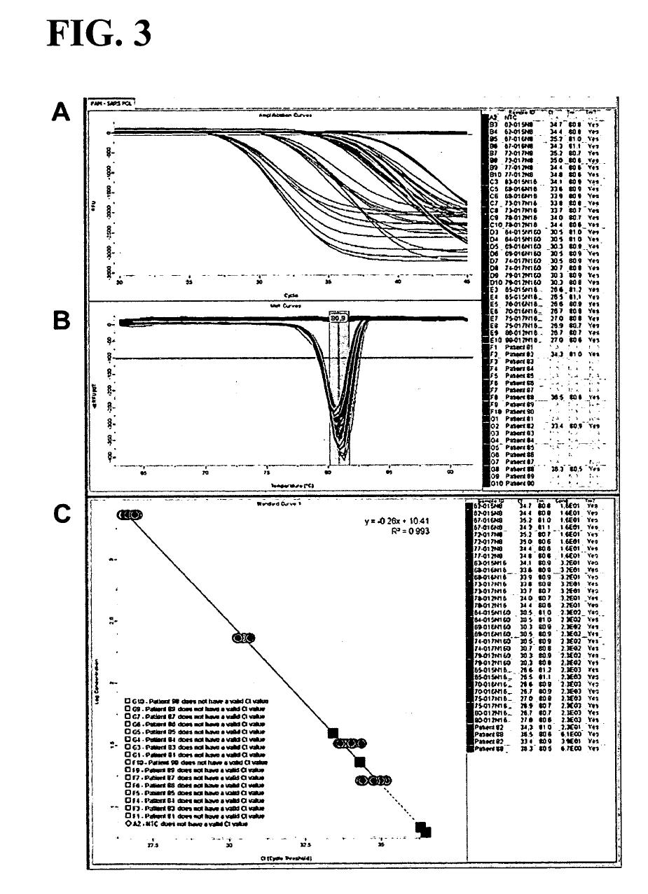

A ten-fold dilution series of SARS viral RNA was prepared from 10⁻¹ PFU/mL to 10⁻¹⁰ PFU/mL in 10 mM MOPS pH 7.4, 0.1 mM EDTA and analyzed using the SARS POL system on the ABI 7900 real-time PCR instrument along with the SARS DNA control (SEQ. ID 12). Limit of detection was found to be 10⁻⁷ PFU/mL. FIG. 2A shows real-time curves for dilutions 10⁻¹ to 10⁻⁷. FIG. 2B shows a melt curve analysis for the same dilution series, confirming amplicon identity.

Table 1 shows cycling threshold (“Ct”) and melt-curve temperature (“Tm”) for each dilution, as well as for a POL DNA control.

TABLE 1

| [Target] (PFU/mL) | Ct | Tm (° C.) |
|---|---|---|
| 10⁻¹ | 16.04 | 81.5 |
| 10⁻² | 20.99 | 81.5 |
| 10⁻³ | 25.06 | 81.2 |
| 10⁻⁴ | 28.23 | 81.2 |
| 10⁻⁵ | 31.64 | 81.2 |
| 10⁻⁶ | 34.76 | 81.2 |
| 10⁻⁷ | 38.45 | 81.2 |
| POL DNA | 28.12 | 80.9 |

11. SARS Detection from Spiked Urine Samples and Patient Samples

Four SARS-negative urine specimens were spiked with SARS viral RNA at four concentrations of 16, 32, 230 and 2300 pfu/mL and were extracted with Qiagen Mini Viral RNA kit (QIAGEN, Valencia, Calif.). The sixteen RNA samples were analyzed in duplicate with the EraGen SARS POL assay on the ABI 7000 real-time PCR instrument. Real-time PCR cycle threshold data was used to construct a standard curve of log SARS concentration vs. Cycle Threshold. Additionally, a linear, least squares fit was performed and the assay was found to be linear with a coefficient of correlation R2=0.993.

RNA extracted from ten potential SARS patient urine specimens was tested in duplicate on the same run as the spiked standards. Two specimens from different patients were found to contain detectable levels of SARS, estimated to be 23-39 pfu/mL and 6-7 pfu/mL respectively (based on the standard curve).

Post-PCR melt analysis confirmed the melting temperature of the SARS-specific amplification product from the positive patient specimens to be the same as that of the spiked standards.

It will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

In addition, where features or aspects of the invention are described in terms of Markush groups or other grouping of alternatives, those skilled in the art will recognize that the invention is also thereby described in terms of any individual member or subgroup of members of the Markush group or other group.

Also, unless indicated to the contrary, where various numerical values are provided for embodiments, additional embodiments are described by taking any 2 different values as the endpoints of a range. Such ranges are also within the scope of the described invention.

All references, patents, and/or applications cited in the specification are incorporated by reference in their entireties, including any tables and figures, to the same extent as if each reference had been incorporated by reference in its entirety individually.

TABLE 2

Exemplary Primers and Control Sequences with
SEQ ID NOs.

| | | |
|---|---|---|
| Target | Sequence (5′→3′) | SEQ ID NO. |

| | | |
|---|---|---|
| SARS-CoV POL | FAM-XAT CAC CCG CGA | SEQ. ID 4 |
| Forward DNA | AGA AGC TAT TC | |
| SARS-CoV POL | AGC CCT CTA CAT CAA | SEQ. ID 5 |
| Reverse DNA | AGC CAA T | |
| SARS-CoV NP | FAM-XCA AAG ACA ACG | SEQ. ID 6 |
| Forward DNA | TCA TAC TGC T | |
| SARS-CoV NP | TTT TGT CCT TTT TAG | SEQ. ID 7 |
| Reverse DNA | GCT CTG TT | |
| SARS-CoV P65 | FAM-XCA AGG GAA AGC | SEQ. ID 8 |
| Forward DNA | CCG TAA AAG | |
| SARS-CoV P65 | AGC CTG TGA GGG AAA | SEQ. ID 9 |
| Reverse DNA | ACC A | |
| Internal Control | HEX-TXG CCT GCT GTG | SEQ. ID 10 |
| Forward DNA | CTG TGT | |
| Internal Control | TCG TGC GGT GCG TC | SEQ. ID 11 |
| Reverse DNA | | |
| POL control | ATC ACC CGC GA GA GCT | SEQ. ID 12 |
| DNA | ATT CGT CAC GTT CGT | |
| | GCT TGG ATT GGC TTT | |
| | GAT GTA GAG GGC T | |
| NP control DNA | CAA AGA CAA CGT CAT | SEQ. ID 13 |
| | ACT GCT GAA CAA GCA | |
| | CAT TGA CGC ATA CAA | |
| | AAC ATT CCC ACC AAC | |
| | AGA GCC TAA AAA GGA | |
| | CAA AA | |
| P65 control DNA | GGG CAA GGG AAA GCC | SEQ. ID 14 |
| | CGU AAA AGG UGC UUG | |
| | GAA CAU UGG ACA ACA | |
| | GAG AUC AGU UUU ACC | |
| | ACC ACU GUG UGG UUU | |
| | UCC CUC ACA GGC U | |
| Internal Control | UCG UGC GGU GCG UCA | SEQ. ID 15 |
| RNA | CAC AGC ACA GCA GGC | |
| Internal Control | UGC AUC CAA CGC GUU | SEQ. ID 16 |
| RNA | GGG AGC UCU CCC AUA | |
| | UGG UCG ACC UGC AGG | |
| | CGG CGC ACU AGU GAU | |
| | ACG CUG CUG UGC UGU | |
| | GUG ACG CAC CGC ACG | |
| | AAA UCC CGC GGC CAU | |
| | GGC GGC CGG GAG CAU | |
| | GCG ACG UCG GGC CCA | |
| | AUU CGC CC | |

FAM: 6-carboxy-fluorescein
HEX: hexachlorofluorescein
Cy5: Cyanine 5

X: deoxy 5-methyl isocytidine

TABLE 3
Exemplary Reaction Formulations: POL and NP

| | | | | For 16 Samples |
|---|---|---|---|---|
| | Concen- | | Per Rxn. | including 10% |
| Component | tration | Final Conc. | (μl) | overage (μl) |
| 2X Reaction | 2x | 1x | 12.5 | 220 |
| Buffer | | | | |
| 50X FAM/HEX | 50x | 1x | 0.5 | 8.8 |
| SARS Primer | | | | |
| Mix | | | | |
| 250 mM DTT | 250 mM | 5 mM | 1.25 | 8.8 |
| Reverse | 25 U/μl | 0.5 U/μl | 0.5 | 8.8 |
| Transcriptase | | | | |
| Titanium Taq | 50x | 1x | 0.5 | 8.8 |
| 25 mM MgCl₂ | 25 mM | 4 mM POL; | 4 POL | 70.4 POL |
| | | 2 mM NP | 2 NP | 35.2 NP |
| Internal | N/A | N/A | 0.5 | 8.8 |
| Control RNA | | | | |
| Nuclease Free | N/A | N/A | 2.25 POL | 39.6 POL |
| Water | | | 0.25 NP | 4.4 NP |
| Total | | | 20 | 352 |
| Reaction | | | | |
| Mix | | | | |
| Volume | | | | |
| Total | | | 25 (w/5 μl | 25 (20 μl rxn. |
| Reaction | | | Target) | mix per tube + |
| Volume | | | | 5 μl Target) |

TABLE 4
Exemplary Reaction Formulations: P65

| | | | | For 16 Samples |
|---|---|---|---|---|
| | | Final | Per Rxn. | including 10% |
| Component | Concentration | Conc. | (μl) | overage (μl) |
| 2X Reaction | 2x | 1x | 12.5 | 220 |
| Buffer | | | | |
| 20X | 20x | 1x | 1.25 | 22 |
| FAM/ROX | | | | |
| SARS Primer | | | | |
| Mix | | | | |
| 100 mM DTT | 100 mM | 5 M | 1.25 | 22 |
| 25 mM MgCl₂ | 25 mM | 3 mM | 3.0 | 52.8 |
| Reverse | 200 U/μl | 0.5 U/μl | 0.06 | 1.06 |
| Transcriptase | | | | |
| Titanium Taq | 50x | 1x | 0.5 | 8.8 |
| Internal | N/A | N/A | 1.0 | 17.6 |
| Control RNA | | | | |
| Nuclease Free | N/A | N/A | 0.44 | 7.74 |
| Water | | | | |
| Total | | | 20 | 352 |
| Reaction | | | | |
| Mix | | | | |
| Volume | | | | |
| Total | | | 25 (w/5 μl | 25 (20 μl rxn. mix |
| Reaction | | | Target) | per tube + 5 μl |
| Volume | | | | Target) |

TABLE 5
Exemplary Reaction Buffer

| Component | 1X Concentration |
|---|---|
| Bis-Tris Propane pH 9.1 | 10 mM |
| Bovine Serum Albumin | 300 μg/ml |
| Tween-20 | 0.4% |
| Potassium chloride | 40 mM |
| MgCl₂ | 2 mM |
| Raffinose | 300 mM |
| DNA polymerase | 7.5 U/rxn |
| dATP | 50 μM-100 μM |
| dGTP | 50 μM-100 μM |
| dCTP | 50 μM-100 μM |
| dTTP | 50 μM-100 μM |
| Primer(s) | 200 μM |
| Reporter (e.g., dabcyl-labeled | 3.0 μM |
| diGTP) | |

What is claimed is:

1. A method for identifying a SARS virus in a sample comprising:
(a) reacting a reaction mixture, the mixture comprising:
(i) the sample;
(ii) at least one oligonucleotide comprising at least one non-natural base, wherein the oligonucleotide is capable of specifically hybridizing to SARS virus nucleic acid; and
(b) detecting the SARS virus nucleic acid if present in the sample.

2. The method of claim 1, wherein the SARS virus is the SARS-associated coronavirus encoded by Genbank Accession No. NC_{—}004718.

3. The method of claim 1, wherein the reaction mixture further comprises:
(iii) internal control nucleic acid; and
(iv) at least one oligonucleotide capable of specifically hybridizing to the internal control nucleic acid;
and the method further comprises:
(c) detecting the internal control nucleic acid.

4. The method of claim 1 wherein the at least one oligonucleotide is capable of specifically hybridizing to a SARS nucleic acid sequence selected from the group consisting of SARS virus nucleoprotein nucleic acid sequence, SARS virus polymerase nucleic acid sequence, SARS virus P65 nucleic acid sequence, and a combination thereof.

5. The method of claim 1, wherein the at least one oligonucleotide comprises a nucleic acid sequence selected from the group consisting of SEQ ID NOs: 4-9.

6. The method of claim 1, further comprising performing RT-PCR of SARS virus RNA.

7. The method of claim 6, further comprising determining a melting temperature of the amplified SARS nucleic acid.

8. The method of claim 1, wherein the reaction mixture comprises at least two oligonucleotides capable of specifically hybridizing to SARS virus nucleic acid and the method further comprises amplifying SARS virus nucleic acid using the two oligonucleotides as primers.

9. The method of claim 8, wherein at least one of the two oligonucleotides used as primers includes a label.

10. The method of claim 9, wherein at least one of the two oligonucleotides used as primers includes at least one base other than A, C, G, T, and U, and wherein the base is selected from iC and iG.

11. The method of claim 10, wherein the label comprises a fluorophore and the reaction mixture further comprises to a quencher covalently linked to the iC or iG.

12. The method of claim 1, wherein the method detects SARS virus nucleic acid that is present in the sample as no more than about 10 copies.

13. A method for detecting SARS virus in a sample comprising:
(a) reacting a mixture that comprises:
(i) the sample;
(ii) a first pair of oligonucleotides capable of specifically hybridizing to SARS virus nucleic acid, wherein at least one oligonucleotide of the first pair includes a first label;
(iii) control nucleic acid; and
(iv) a second pair of oligonucleotides capable of specifically hybridizing to the control nucleic acid, wherein at least one oligonucleotide of the second pair includes a second label, and the first label and second label are different;
(b) amplifying and detecting the control nucleic acid and the SARS virus nucleic acid, if present in the sample.

14. The method of claim 13, wherein the SARS virus is the SARS-associated CoV encoded by the nucleic acid sequence deposited as Genbank Accession No. NC_{—}004718.

15. The method of claim 13, wherein the first pair of oligonucleotides is capable of specifically hybridizing to nucleic acid selected from the group consisting of nucleic acid of SARS virus polymerase, nucleic acid of SARS virus nucleoprotein, nucleic acid of SARS virus P65 nucleic acid sequence, and combinations thereof.

16. The method of claim 13, wherein at least one oligonucleotide of the first pair of oligonucleotides includes at least one base other than A, C, G, T, and U, and wherein the base is selected from iC and iG, and wherein at least one oligonucleotide of the second pair of oligonucleotides includes at least one base other than A, C, G, T, and U, and wherein the base is selected from iC and iG.

17. The method of claim 13, wherein the first label and second label comprise two different fluorophores and the reaction mixture further comprises a nucleotide covalently linked to a quencher that is capable of quenching the two different fluorophores.

18. The method of claim 13, wherein the method detects SARS virus nucleic that is present in the sample as no more than about 10 copies.

19. A kit comprising:
(a) a first pair of oligonucleotides capable of specifically hybridizing to a SARS virus nucleic acid, wherein at least one oligonucleotide of the first pair comprises at least one non-natural base and a label.

20. The kit of claim 19, further comprising:
(b) control nucleic acid; and
(c) a second pair of oligonucleotides capable of specifically hybridizing to the control nucleic acid, wherein at least one oligonucleotide of the second pair comprises at least one non-natural base and a second label;
wherein the first label and second label are different;
(d) a nucleotide that includes a non-natural base that base-pairs with the non-natural base present in the at least one oligonucleotide of the first pair and the non-natural base present in the at least one oligonucleotide of the second pair.
